SEQUENCE LISTING (4 sequences)

SEQ ID NO: 1 - DNA, 34 bp, artificial sequence
  Description of Artificial Sequence Synthetic oligonucleotide
  misc_feature (34)..(34): n is u
tggttttgcctgtcgtgttgtctcgtgctcttcn

SEQ ID NO: 2 - DNA, 33 bp, artificial sequence
  Description of Artificial Sequence Synthetic oligonucleotide
gaagagcacgagaaggagaagagcaacggcaag

SEQ ID NO: 3 - DNA, 34 bp, artificial sequence
  Description of Artificial Sequence Synthetic oligonucleotide
  misc_feature (34)..(34): n is u
cttgccgttgctcttctccttctcgtgctcttcn

SEQ ID NO: 4 - DNA, 33 bp, artificial sequence
  Description of Artificial Sequence Synthetic oligonucleotide
  misc_feature (33)..(33): n is u
ggttttgcctgtcgtgttgtctcgtgctcttcn

(19) United States
(12) Patent Application Publication
(10) Pub. No.: US 2022/0348996 A1
(43) Pub. Date: Nov. 3, 2022

(54) METHOD AND KIT FOR NON-SPECIFIC AMPLIFICATION OF NATURAL SHORT-FRAGMENT NUCLEIC ACID

(71) Applicant: BERRY GENOMICS CO., LTD, Beijing (CN)

(72) Inventors: Jie Tian, Beijing (CN); Nalahu Wu, Beijing (CN); Xueyan Yang, Beijing (CN); Yang Zhang, Beijing (CN); Jianguang Zhang, Beijing (CN)

(21) Appl. No.: 17/632,404

(22) PCT Filed: Jul. 31, 2020
(86) PCT No.: PCT/CN2020/106373

§ 371 Date: Feb. 2, 2022

(30) Foreign Application Priority Data
Aug. 2, 2019 (CN) 201910712493.4

(51) Int. Cl. C12Q 1/6844; C12Q 1/6806

(52) U.S. Cl. CPC C12Q 1/6844; C12Q 1/6806

(57) ABSTRACT

The present invention relates to a method for a non-specific amplification of a natural short-fragment nucleic acid, comprising the following steps: (1) performing end repair on the natural short-fragment nucleic acid to obtain an end-repaired nucleic acid; (2) connecting the end-repaired nucleic acid to a double-stranded linker to obtain a ligation product, in which each strand of the double-stranded linker contains only three bases; (3) performing PCR amplification on the ligation product using a PCR primer labeled with deoxyuridine to obtain a PCR product, in which the PCR primer is completely or partially complementary to a strand of the double-stranded linker and contains only three bases; and (4) digesting the PCR product by using an enzyme having a deoxyuridine cleavage function, followed by digesting the PCR product by using an enzyme with both 5′→3′ polymerase activity and 3′→5′ exonuclease activity in the presence of a deoxynucleotide solution to obtain a non-specific amplification product of the natural short-fragment nucleic acid. The deoxynucleotide solution only contains the complementary base of the base lacking in the primer. The present invention also relates to a kit for implementing the aforementioned method.

### TECHNICAL FIELD

The present invention belongs to the field of molecular biology, specifically relates to a method and a kit for non-specific amplification of natural short-fragment nucleic acid.

### BACKGROUND

Natural short-fragment nucleic acids are extracellular free DNAs (cell-free DNAs, cDNAs) existing in animal, plant, and human body fluid, typically is less than 500 bp in length. During apoptosis, DNAs in human cells are cleaved and secreted extracellularly to form cfDNAs. Currently, the use of cfDNAs as a marker has been demonstrated in studies of tumor and prenatal diagnostic. However, in one aspect, the amount of cfDNAs in body fluid is very low, for example, in plasma, even lower than 10 ng/mL, and loss of cfDNAs tends to occur during extraction, resulting in a difficult process of extraction. cfDNAs, in another aspect, should be used in a huge amount in fluid biopsy and non-invasive prenatal screening. These make it difficult for the directly extracted natural short-fragment nucleic acids to fulfill the requirements of the test, standard, quality control and reference substances in terms of amount and quality.

Currently, in some methods, long-fragment DNAs are ultrasonicated to obtain DNA fragments similar to natural short-fragment nucleic acids in size. However, the DNA fragment thus obtained is inherently different from the natural short-fragment nucleic acid, e.g. in size distribution. In addition, in some methods the short-fragment DNA is obtained through enzymatic digestion. However, the length distribution of enzymatic digestion products is also significantly different from that of the natural short-fragment nucleic acid, and limited by enzymatic digestion site. Generally, it is difficult for enzymatic digestion product to have the size of the natural short-fragment nucleic acid (for example, the average length of plasma cfDNA is only about 170 bp), and the ends thereof are less variable than the natural short-fragment nucleic acid. Therefore, neither ultrasonicated nor enzymatically digested DNA fragment is able to replace the natural short-fragment nucleic acid as the test, standard, quality control and reference substances.

Therefore, a method capable of obtaining large amounts of natural short-fragment nucleic acids is in need to address the issue of research and testing for such DNAs.

### SUMMARY

In response to the above-mentioned demand, the present invention provides a method for non-specific amplification of natural short-fragment nucleic acids. The method efficiently amplifies the natural short-fragment nucleic acid as starting material using a specifically designed linker and primer, to obtain large quantities of DNA fragments substantially identical to the natural short-fragment nucleic acid in both DNA sequence and fragment length distribution. The present invention also provides a kit for non-specific amplification of the natural short-fragment nucleic acids.

The basic principle of the present invention is: a double-stranded linker containing only three types of bases in each strand is attached to both ends of the natural short-fragment nucleic acids, then the linker-added DNA fragments are amplified with a deoxyuridine-labeled primer by using PCR technique, and then the PCR amplification product is enzymatically digested using an enzyme having a deoxyuridine cleavage function and an enzyme with both 5′→3′ polymerase activity and 3′→5′ exonuclease activity successively to completely remove the introduced linker sequence, thereby obtaining large quantities of DNA fragments substantially identical to the natural short-fragment nucleic acids in terms of DNA sequence and fragment length distribution.

Accordingly, in a first aspect, the present invention provides a method for non-specific amplification of a natural short-fragment nucleic acid comprising the following steps:

(1) performing end repair on the natural short-fragment nucleic acid to obtain an end-repaired nucleic acid;

(2) connecting the end-repaired nucleic acid to a double-stranded linker to obtain a ligation product, in which each strand of the double-stranded linker contains only three types of bases;

(3) performing PCR amplification on the ligation product using a PCR primer labeled with deoxyuridine to obtain a PCR product, in which the PCR primer is completely or partially complementary to a strand of the double-stranded linker and contains only three types of bases; and

(4) digesting the PCR product by using an enzyme having a deoxyuridine cleavage function, followed by digesting the PCR product by using an enzyme with both 5′→3′ polymerase activity and 3′→5′ exonuclease activity in the presence of a deoxynucleotide solution to obtain a non-specific amplification product of the natural short-fragment nucleic acid; the deoxynucleotide solution only contains the complementary base of base lacking in the PCR primer.

In one embodiment, the natural short-fragment nucleic acid is a double-stranded DNA of less than 500 bp.

In one embodiment, the origins of the natural short-fragment nucleic acid include, but not limited to, blood, serum, plasma, joint fluid, semen, urine, sweat, saliva, stool, cerebrospinal fluid, ascites, pleural effusion, bile, pancreatic fluid, and the like. Preferably, the natural short-fragment nucleic acid is derived from plasma, blood, or urine.

In one embodiment, the end repair employs one or more enzymes selected from the group consisting of: T4 DNA polymerase, klenow enzyme, T4 polynucleotide kinase and Klenow Fragment enzyme. Other enzymes known in the art to be useful for end-repair are also suitable for use in the invention.

In one embodiment, said method further comprises the step of addition of poly-adenine at 3′ end of the end-repaired nucleic acid prior to being connected to the linker. In this embodiment, the steps of end repair and addition of poly-adenine at 3′ end could be performed in one reaction system, i.e., after the end repair, the poly-adenine should be added at 3′ end after purification. Alternatively, and preferably, the steps of end repair and addition of poly-adenine at 3′ end are performed in one reaction system, i.e., end-repair and addition of poly-adenine at 3′ end are completed simultaneously followed by the purification of the nucleic acid. Alternatively, and more preferably, end repair, addition of poly-adenine at 3′ end, and linker ligation are performed in one reaction system, and the ligation reaction is followed by purification of the nucleic acid for PCR amplification. In this embodiment, the step of addition of poly-adenine at 3′ end may employ klenow ex-enzyme, Taq enzyme, or a combination of klenow ex-enzyme and Taq enzyme. Other enzymes known in the art to be useful for adding poly-adenine at 3′ end are also suitable for use in the present invention.

In one embodiment, each strand forming the double-stranded linker comprises only any three types of bases, e.g., only A/C/T, A/C/G, C/T/G, or A/T/G. Each strand may contain combinations of bases different from each other, for example one strand may contain A/C/T and the other one A/C/G. Preferably, two strands of the double-stranded linker are completely or partially reverse complementary. In one embodiment, 5′ end of one of the strands forming the double-stranded linker carries phosphate groups. More preferably, in both strands forming the double-stranded linker, 3′ end of one strand is deoxyuridine U and 5′ end of the other strand carries phosphate groups. In that case, the presence of deoxyuridine in the double-stranded linker facilitates cleavage of the linker linked to the natural short-fragment nucleic acid as template, thereby increasing the yield of non-specific amplification; in other words, the final product obtained in that case still contains a copy of the natural short-fragment nucleic acid as template.

For instance, the linker is formed by annealing of the single-stranded DNAs shown in the following SEQ ID NO: 1 and SEQ ID NO: 2:

SEQ ID NO: 1:
5′-TGGTTTTGCCTGTCGTGTTGTCTCGTGCTCTTCU-3′
SEQ ID NO: 2:
5′-GAAGAGCACGAGAAGGAGAAGAGCAACGGCAAG-3′

In one embodiment, the linker ligation is performed by T4 DNA ligase and/or T7 DNA ligase.

In one embodiment, a PCR amplification primer that is completely or partially complementary to one strand of the double-stranded linker has a deoxyuridine label and comprises only three types of bases. The deoxyuridine label contained in the primer allows a single base notch formed between the primer and the target nucleic acid upon subsequent enzymatic digestion with an enzyme having a deoxyuridine cleavage function, thereby completely removing the primer sequence. For example, the primers have the following sequences:

SEQ ID NO: 3:
5′-CTTGCCGTTGCTCTTCTCCTTCTCGTGCTCTTCU-3′
SEQ ID NO: 4:
5′-GGTTTTGCCTGTCGTGTTGTCTCGTGCTCTTCU-3′

In one embodiment, the PCR product is digested with an enzyme having a deoxyuridine cleavage function followed by digestion with an enzyme having both 5′→3′ polymerase activity and 3′→5′ exonuclease activity in the presence of deoxynucleotide solution to remove primer sequence and linker in PCR product; wherein the enzymes having deoxyuridine cleavage function include, but not limited to: USER™ enzymes, and a mixture comprising Endonuclease VIII and UDG; and the enzymes having both 5′→3′ polymerase activity and 3′→5′ exonuclease activity include, but not limited to, T4 DNA polymerase.

In one embodiment, the deoxynucleotide solution contains merely complementary base of the base lacking in the PCR primer. For example, when the double PCR primers contain only A/C/T (i.e., it lacks G), the added deoxynucleotide solution only contains base C complementary to G.

For instance, T4 DNA polymerase possesses both 5′→3′ polymerase activity and 3′→5′ exonuclease activity, and substantially exerts 5′→3′ polymerase activity in the presence of adequate dNTPs contained in reaction substrate, while mainly 3′→5′ exonuclease activity in case of insufficient dNTPs in reaction substrate. Since the PCR primer contains only three types of bases, no base complementary to the primer is present in the system at the beginning of the reaction, allowing T4 DNA polymerase to perform 3′→5′ exonuclease function. When the linker/primer is completely cleaved on both ends and lacking base of the above primer appears in sequence, the base contained in the deoxynucleotide solution added in the system is capable of being complementary to said lacking base, resulting in T4 DNA polymerase exerts 5′→3′ polymerase activity, thereby achieving an equilibrium of the reaction, stopping an enzymatic digestion reaction, and obtaining a product with substantially identical to the original cfDNA fragments in size.

In the second aspect, the invention provides a kit for non-specific amplification of a natural short-fragment nucleic acid comprising:

(1) a reagent for performing end repair;

(2) a reagent for connecting an linker comprising a double-stranded linker, wherein each strand of the double-stranded linker comprises only three types of bases;

(3) a reagent for PCR amplification comprising a PCR primer with a deoxyuridine label, wherein the PCR primer is completely or partially complementary to one strand of the double-stranded linker, and comprises only three types of bases;

(4) a reagent for enzymatic digestion, comprising an enzyme with deoxyuridine cleavage function, an enzyme with both 5′→3′ polymerase activity and 3′→5′ exonuclease activity, and a deoxynucleotide solution containing only the complementary base of the base lacking in the PCR primer.

In one embodiment, the agent for performing end repair comprises one or more enzymes selected from the group consisting of: T4 DNA polymerase, T4 polynucleotide kinase, Klenow Fragment enzyme and Klenow enzyme.

In one embodiment, the kit further comprises a reagent for adding poly-adensine to 3′ end. Specifically, the reagent for adding poly-adensine to 3′ end comprises klenow ex-enzyme, Taq enzyme, or a combination of klenow ex-enzyme and Taq enzyme.

In one embodiment, the reagent for connecting a linker further comprises T4 DNA ligase and/or T7 DNA ligase.

In one embodiment, each strand of the double-stranded linker comprises only any three types of bases, e.g., only comprises A/C/T, A/C/G, C/T/G, or A/T/G. Each strand may contain combinations of bases different from each other, for example one strand may contain A/C/T and the other one A/C/G. In one embodiment, preferably, two strands of the double-stranded linker are completely or partially reverse complementary. In one embodiment, 5′ end of one of the strands forming the double-stranded linker carries phosphate groups. More preferably, in the two single-stranded DNA forming linker, the 3′ end of one strand is deoxyuridine U and the 5′ end of the other strand carries phosphate groups.

In one embodiment, the kit of the invention may further comprise reagents for purification.

In one embodiment, the enzymes having deoxyuridine cleavage function include, but not limited to: USER™ enzyme, and a mixture comprising Endonuclease VIII and UDG. In another embodiment, the enzymes with both 5′→3′ polymerase activity and 3′→5′ exonuclease activity include, but not limited to, T4 DNA polymerase.

In one embodiment, the reagent for enzymatic digestion comprises deoxynucleotide solution containing merely the complementary base of the base lacking in the PCR primer. For example, when the PCR primer contains only A/C/T (i.e., it lacks G), the added deoxynucleotides solution contains only base C complementary to G.

In the kit of the invention, each reagent or device is preferably packaged separately, or may be packaged in combination without affecting the practice of the invention.

In a third aspect, the invention also relates to a non-specifically amplified natural short-fragment nucleic acid obtained using the method or kit of the invention, their use as the test, standard, control or reference substances, and composition comprising the same.

The excellent technical effects of the present invention are as follows: a non-destructive amplification of natural short-fragment nucleic acid was achieved by the non-specific amplification method, which effectively reduced the preference of enzyme digestion and damage to DNAs. DNA fragments mimicking the natural short-fragment nucleic acid can be prepared in large quantities, which are basically identical to natural short-fragment nucleic acid in terms of sequence and length distribution. In addition, the method of the present invention enables an efficient enrichment of fetal-derived DNAs in cfDNAs with a non-destructive amplification, allowing for increasing content of fetal DNAs in a non-invasive pre-natal testing, thereby improving detection sensitivity and accuracy.

The invention will now be described in more detail by way of examples with reference to the accompanying drawings. It should be understood by those skilled in the art that the drawings and examples thereof are for illustrative purposes only and not to be construed as limiting the invention in any way. The embodiments and features thereof in the present application can be combined with each other without contradiction.

### BRIEF DESCRIPTION OF THE FIGURE

FIG. 1: a size distribution of natural plasma free DNAs, the non-specifically amplified fragment thereof and commercially available free DNA standard.

### DETAILED DESCRIPTION

### Example 1: A Non-Specific Amplification of Plasma Free DNAs According to the Method of the Invention

1. Preparation of Plasma Free DNAs

Free DNAs were extracted from peripheral blood of pregnant women by using a plasma free DNA extraction kit (Hangzhou Berrygenomics Diagnostic Technology Co. Ltd. Cat No. R0011) to obtain 4 ng of plasma DNAs.

2. End Repair and Addition of ‘A’s

The NEBNext end repair/dAs tail-adding Module kit (NEB, E7442S) was used to prepare the reaction system shown in Table 1 for simultaneous end repair and treatment of adding poly-adenine at 3′ end. The reaction procedure was as follows: 37° C., 20 min; 72° C., 20 min; stored at 4° C. After completion of the reaction, the next step was conducted without purification.

TABLE 1

| Plasma free DNAs | 4 ng (40.5 μL) | |
|---|---|---|
| End Prep Enzyme Mix | 1.5 | μL |
| End Repair Reaction Buffer (10X) | 3.3 | μL |
| ddH₂O | 3.7 | μL |
| Total volume | 49 | μL |

3. Ligation of Linker

(1) Preparation of Linker

The sequence as shown in Table 2 was synthesized, with SEQ ID NO: 1 having deoxyuridine label at 3′ end and SEQ ID NO: 2 having phosphate group label at 5′ end:

TABLE 2

| SEQ ID NO | 5′→3′ |
|---|---|
| SEQ ID NO: 1 | CTTGCCGTTGCTCTTCTCCTTCTCGTGCTCTTCU |
| SEQ ID NO: 2 | GAAGAGCACGAGAAGGAGAAGAGCAACGGCAAG |

The synthesized sequence dry powder was diluted with sterilized water to a concentration of 100 μM, to 25 μL each was added 20 μL of 5× annealing buffer and 30 μL of sterilized water, mixed evenly, and then annealed.

The annealing procedure was as follows: reaction at 95° C. for 2 min, cooling at 0.1° C./s to 90° C., reaction for 2 min, cooling at 0.1° C./s to 85° C., reaction for 2 min, and so on, until the temperature dropped to 25° C., reaction for 2 min, and stored at 4° C. to obtain 25 μM linker, and diluted to 2.5 μM with linker diluent for further use.

(2) Ligation of the Linker

The linker ligation reaction system shown in Table 3 was prepared using T4 DNA ligase (NEB, M0202L) and the reaction was carried out according to the following procedure: 20° C., 15 min; 65° C., 10 min; stored at 4° C. After completion of the reaction, 40 μL of L AMPure XP Beads was added for recovery and then eluted with 26 μL of EB to give the ligation product.

TABLE 3

| | | |
|---|---|---|
| Reaction system of Step 2 | 49 | μL |
| Connection reaction buffer | 25 | μL |
| 2.5 μM linker | 0.75 | μL |
| T4 DNA ligase | 1 | μL |
| Sterilized water | 2.25 | μL |
| Total volume | 78 | μL |

4. PCR Amplification

(1) Preparation of Primers

The primer sequences shown in Table 4 were synthesized, with both SEQ ID NO: 3 and SEQ ID NO: 4 having deoxyuridine label at 3′ end:

TABLE 4

| SEQ ID NO | 5′→3′ |
|---|---|
| SEQ ID NO: 3 | CTTGCCGTTGCTCTTCTCCTTCTCGTGCTCTTCU |
| SEQ ID NO: 4 | GGTTTTGCCTGTCGTGTTGTCTCGTGCTCTTCU |

The synthesized primers dry powder was diluted with sterilized water to the concentration of 10 μM for use.

(2) PCR Amplification

The amplification reaction system shown in Table 5 was prepared using KAPA2G Robust HotStart PCR Kit (KAPA, KK5517) and the reaction procedure was as follows: 98° C., 30 s; 98° C. 10 s, 62° C. 30 s, 72° C. 30 s, 20 cycles; 72° C. 5 minutes; stored at 4° C.

TABLE 5

| | | |
|---|---|---|
| Ligation product | 23 | μL |
| 5X KAPA2G Buffer A | 10 | μL |
| 10 mM dNTP Mix | 1 | μL |
| 10 μM Primer SEQ ID NO: 3 | 2.5 | μL |
| 10 μM Primer SEQ ID NO: 4 | 2.5 | μL |
| KAPA2G Robust DNA polymerase | 0.2 | μL |
| EB | 10.8 | μL |
| Total volume | 50 | μL |

After the reaction was complete, 60 μL AMPure XP Beads was added for purification followed by elution with 50 μL of EB to yield PCR amplification product.

5. Enzymatic Digestion Reaction

The reaction system set forth in Table 6 was prepared and the amplification product was digested into notched DNA fragment using USER™ enzyme (NEB, M5508).

TABLE 6

| | | |
|---|---|---|
| USER ™ Digestion Buffer (10×) | 5 | μL |
| USER ™ Enzyme | 1 | μL |
| PCR amplification product | 200 | ng |
| Sterilized water | ~44 | μL |
| Total volume | 50 | μL |

The reaction procedure was as follows: 37° C., 30 min; 65° C., 10 min; stored at 4° C. After completion of the reaction, the next step was carried out without purification.

The reaction set forth in Table 7 was then prepared and the notched DNA fragment was treated with T4 DNA polymerase (NEB, M0203L) to completely cleave the linker sequence.

TABLE 7

| | | |
|---|---|---|
| Notched DNA fragment | 50 | μL |
| Polymerase Buffer (10X) | 6 | μL |
| dTTP (10 mM) | 1 | μL |
| EB | 2 | μL |
| Total volume | 59 | μL |

The above system was incubated for 5 minutes at 70° C. and stopped, and T4 DNA polymerase was added and incubated for an additional 5 minutes at 37° C. for digestion. The enzyme was inactivated by vigorous vibration at end of the reaction. Further purification by addition of 90 μL AMPure XP Beads followed by elution with 50 μL EB yielded a non-specific amplification product with a yield of 80 ng, a 20-fold increase compared to the concentration of original plasma DNA.

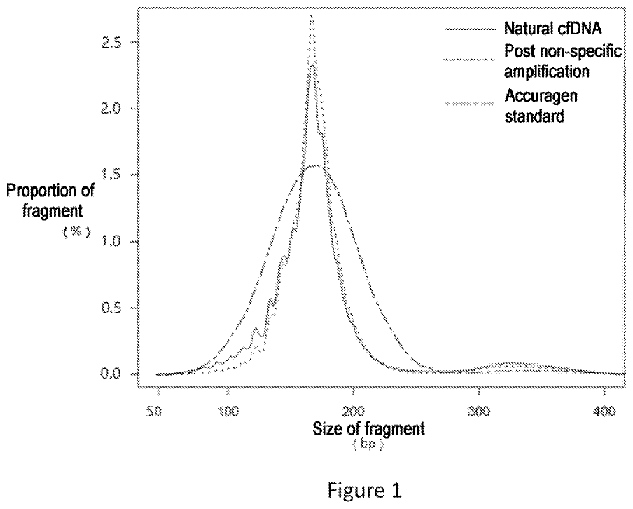

With fetal chromosomal aneuploidy (T13/T18/T21) detection kit (Hangzhou Berrygenomics Diagnostic Technology Co. Ltd. Cat No. R0000), library construction and high-throughput sequencing were performed using directly extracted original plasma DNAs (i.e. natural cfDNAs), commercially available free DNAs standard (Accuragen, cat No. AG-STD-S-KA-8) and the non-specific amplification product obtained according to method of the present invention, respectively, and the data generated by double-end sequencing was compared and analyzed. The obtained fragment distribution statistics are shown in FIG. 1.

As can be seen from FIG. 1, the DNA product after the non-specific amplification by the method of the present invention is consistent in fragment distribution with the original natural plasma DNAs and is able to mimic the state of natural plasma DNAs than the commercially available free DNAs standard more accurately.

### Example 2: Non-Specific Amplification of Tumor Plasma Free DNAs According to the Method of the Invention

The steps of Example 2 were as those of Example 1, except for the original DNA sample. Specifically, tumor plasma free DNAs were extracted with agMAX Cell-Free DNA Isolation Kit (Thermo Fisher, Cat No. A29319) to obtain free DNAs in a total amount of 6 ng of DNAs. The yield was 132 ng after the non-specific amplification, a 22-fold increase compared to the concentration of original plasma DNA.

### Example 3: Quality Assessment of Non-Specific Amplified Natural Short-Fragment Nucleic Acids

4 ng of natural plasma free DNAs and the non-specifically amplified product prepared according to method of Example 1 were taken, respectively, and according to the manufacturer's instructions, a sequencing library was constructed using the fetal chromosomal aneuploidy (T13/T18/T21) detection kit (Beijing Berrygenomics, Cat No. R1000). Sequencing was then performed using Nextseq CN500 sequencer with the sequencing type of 36SE and 5M data obtained for each sample. The quality of the library sequencing data is shown in Table 8, wherein Total Rds is the total number of sequences detected, Uniq % is the percentage of DNA sequences uniquely aligned to human genome (hg19) in Total Rds, Redundancy % is the percentage of redundant reads in Total Rds, and Unimap_GC % is the GC percentage of DNA sequences uniquely aligned to human genome (hg19)

TABLE 8

| | | Uniq | Redundancy | UniMap_GC |
|---|---|---|---|---|
| | Total_Rds | % | % | % |
| Non-specific | 5884689 | 61.89 | 1.32 | 40.08 |
| Amplification | | | | |
| Product of | | | | |
| Example 1 | | | | |
| Natural plasma | 6671223 | 66.14 | 0.99 | 39.32 |
| free DNA | | | | |

As can be seen from Table 8, Total Rds, Uniq %, Redundancy %, and UniMap_GC % of the non-specific amplification product prepared according to Example 1 of the present invention all have achieved the required amount of data and close to natural plasma free DNAs.

The sequencing data was analyzed for chromosomal aneuploidy using analysis software and the result was shown in Table 9, wherein Chr13 Z value is the Z value of chromosome 13 of the sample, i.e., the deviation between the percentage of bases detected on chromosome 13 in all the detected bases of the sample and the percentage of bases on chromosome 13 of normal samples in all the bases in parameter database. Chr18 Z value and the Chr21 Z value are the Z values of chromosome 18 and chromosome 21, respectively. Samples with Z values between [−3.00, 3.00] are normal ones. The Cff % content is the fetal DNAs content in free DNAs.

TABLE 9

| | Chr13 | Chr18 | Chr21 | Cff % |
|---|---|---|---|---|
| Sample information | Z Value | Z Value | Z Value | Content |
| Non-specific Amplification | 1.41 | 0.65 | −1.34 | 8.17% |
| Product of Example 1 | | | | |
| Natural plasma free DNA | 0.32 | 1.19 | 0.56 | 6.35% |

As can be seen from Table 9, Chr13 Z value, Chr18 Z value and Chr21 Z value of the non-specific amplification product prepared according to Example 1 of the present invention and the natural plasma free DNA samples are all between [−3.00, 3.00], the detection results are consistent, and the sample is negative. Furthermore, the content of fetal DNAs in the non-specific amplification product obtained by the method of the present invention is up to 8.17%, an increase of 28.66% compared to original sample (6.35%).

Thus, the non-specific amplification product prepared according to Example 1 of the present invention was substantially identical in sequence to natural plasma free DNA samples. While nondestructively amplifying natural plasma free DNAs, the method of the present invention also significantly enriched the DNAs derived from fetus, which is advantageous for improving detection sensitivity and accuracy in prenatal diagnosis.

### Example 4: Quality Assessment of Non-Specifically Amplified Natural Short-Fragment Nucleic Acids

5 ng of natural tumor plasma free DNAs and 5 ng of non-specifically amplified product prepared according to method of Example 2 were taken, respectively, and according to the manufacturer's instructions, a sequencing library was constructed using cSMART tumor gene mutation detection kit 1 (10 genes) (Beijing Berrygenomics. Cat No. R0024). Sequencing was then performed using Nextseq CN 500 sequencer with the sequencing type of 150PE and 10M data obtained for each sample. The quality of the library sequencing data is shown in Table 10, wherein Clean Reads is the percentage of reads after removing redundant sequences, CleanQ30 is the Q30 of Clean Reads sequence, and MeanDepth is average sequencing depth.

TABLE 10

| Sample information | Clean Reads | CleanQ30 | MeanDepth |
|---|---|---|---|
| Non-specific | 11552491 | 83.87 | 33600 |
| Amplification | | | |
| Product of | | | |
| Example 2 | | | |
| Natural tumor | 11353273 | 82.92 | 30557 |
| plasma free DNA | | | |

As can be seen from Table 10, Clean Reads, CleanQ30 and MeanDepth of the non-specific amplification product prepared according to Example 2 of the present invention all have achieved the required amount of data and are close to that of natural tumor plasma free DNAs.

SNP and InDel analysis was performed on sequencing data using analysis software, and it is found that the non-specific amplification product prepared by method of the present invention is consistent with natural tumor plasma free DNAs in the proportion of 0.3% gene mutation, and no more than 0.3% of SNP and InDel was detected, indicating that the sequences of the non-specific amplification product prepared according to Example 2 of the present invention is substantially consistent with that of the natural tumor plasma free DNA sample.

### Example 5: Comparison of Different Digestion Systems

PCR amplification product was obtained according to steps 1-4 of the method described in Example 1. 200 ng PCR amplification product was digested with different digestion systems as shown in Table 11 below. After completion of digestion reaction, the product was purified with 90 μL AMPure XP Beads and eluted with 50 μL of EB, and the digestion product contained in eluent was quantified.

TABLE 11

| | | Digestion |
|---|---|---|
| Test | | product content |
| Number | Digestion system | (ng) |
| 1 | USER ™ enzyme followed by T4 DNA | 80 |
| | polymerase | |
| 2 | T4 DNA polymerase only | 0 |
| 3 | T4 DNA polymerase followed by USER ™ | 0 |
| | enzyme | |

Note:
the digestion system and reaction time of USER ™ enzyme was identical to those of T4 DNA polymerase in Tests 1-3, and was the digestion system and reaction time as shown in step 5 of Example 1.

The above results show that the specific digestion reaction used in the method of the present invention could obtain a significantly higher yield with an excellent technical effect, as compared to that where only one enzyme is used for digestion and two enzymes are used for digestion in a reverse order.

It should be noted that the above illustrated is the preferred embodiments of the invention rather than limiting the invention. Various modifications and changes can be made to the present invention for those skilled in the Art. It will be understood by the skilled in the art that any change, equivalent replacement, and improvement made shall be included in the protection scope of the present invention without departing from the concepts and principles of the invention.

1. A method for non-specific amplification of a natural short-fragment nucleic acid, comprising the following steps:
(1) performing end repair on the natural short-fragment nucleic acid to obtain an end-repaired nucleic acid;
(2) connecting the end-repaired nucleic acid to a double-stranded linker to obtain a ligation product, in which each strand of the double-stranded linker contains only three types of bases;
(3) performing PCR amplification on the ligation product using a PCR primer labeled with deoxyuridine to obtain a PCR product, in which the PCR primer is completely or partially complementary to a strand of the double-stranded linker and contains only three types of bases; and
(4) digesting the PCR product by using an enzyme having a deoxyuridine cleavage function, followed by digesting the PCR product by using an enzyme with both 5′→3′ polymerase activity and 3′→5′ exonuclease activity in the presence of a deoxynucleotide solution to obtain a non-specific amplification product of the natural short-fragment nucleic acid;
the deoxynucleotide solution contains the complementary base of the base lacking in the PCR primer.

2. The method of claim 1, wherein the natural short-fragment nucleic acid is a double-stranded DNA of less than 500 bp.

3. The method of claim 1, wherein the natural short-fragment nucleic acid derived from the group consisting of blood, serum, plasma, joint fluid, semen, urine, sweat, saliva, stool, cerebrospinal fluid, ascites, pleural effusion, bile, and pancreatic fluid.

4. The method of claim 3, wherein the natural short-fragment nucleic acid derived from plasma, blood, or urine.

5. The method of claim 1, wherein the end-repair employs one or more enzymes selected from the group consisting of: T4 DNA polymerase, T4 polynucleotide kinase, Klenow Fragment enzyme and Klenow enzyme.

6. The method of claim 1, wherein the method further comprises the step of addition of poly-adenine at 3′ end of the end-repaired nucleic acid prior to being connected to the linker.

7. The method of claim 6, wherein the steps of end repair and addition of poly-adenine at 3′ end are performed in one reaction system.

8. The method of claim 6, wherein the three steps of end repair, addition of poly-adenine at 3′ end, and the linker ligation are performed in one reaction system.

9. The method of claim 6, wherein in the step of addition of poly-adenine at 3′end, klenow ex-enzyme, Taq enzyme, or a combination of klenow ex-enzyme and Taq enzyme is employed.

10. The method of claim 1, wherein two strands of the double-stranded linker are completely or partially reverse complementary.

11. The method of claim 1, wherein the 3′ end of one strand is deoxyuridine.

12. The method of claim 1, wherein the connection in step (2) is performed by T4 DNA ligase and/or T7 DNA ligase.

13. The method of claim 1, wherein the enzyme having deoxyuridine cleavage function is selected from the group consisting of: USER™ enzyme, and a mixture comprising Endonuclease VIII and UDG.

14. The method of claim 1, wherein the enzyme with both 5′→3′ polymerase activity and 3′→5′ exonuclease activity is T4 DNA polymerase.

15. A kit for non-specific amplification of natural short-fragment nucleic acid, comprising:
(1) a reagent for performing end repair;
(2) a reagent for connecting an linker comprising a double-stranded linker, wherein each strand of the double-stranded linker comprises only three types of bases;
(3) a reagent for PCR amplification comprising a PCR primer with a deoxyuridine label, wherein the PCR primer is completely or partially complementary to one strand of the double-stranded linker, and comprises only three types of bases;
(4) a reagent for enzymatic digestion, comprising an enzyme with deoxyuridine cleavage function, an enzyme with both 5′→3′ polymerase activity and 3′→5′ exonuclease activity, and a deoxynucleotide solution containing only the complementary base of the base lacking in the PCR primer.

16. The kit of claim 15, wherein the reagent for performing end repair comprises one or more enzymes selected from the group consisting of: T4 DNA polymerase, T4 polynucleotide kinase, Klenow Fragment enzyme and Klenow enzyme.

17. The kit of claim 15, wherein the kit further comprises a reagent for adding poly-adensine to 3′ end.

18. The kit of claim 17, wherein the reagent for adding poly-adensine to 3′ end comprises klenow ex-enzyme, Taq enzyme, or a combination of klenow ex-enzyme and Taq enzyme.

19. The kit of claim 15, wherein the reagent for connecting a linker further comprises T4 DNA ligase and/or T7 DNA ligase.

20. The kit of claim 15, wherein the two strands of the double-stranded linker are completely or partially reverse complementary.

21. The kit of claim 15, wherein the 3′ end of one strand is deoxyuridine U.

22. The kit of claim 15, wherein the enzyme having deoxyuridine cleavage function is selected from the group consisting of: USER™ enzyme, and a mixture comprising Endonuclease VIII and UDG.

23. The kit of claim 15, wherein the enzyme with both 5′→3′ polymerase activity and 3′→5′ exonuclease activity is T4 DNA polymerase.

24. The kit of claim 15, wherein the kit may further comprise reagents for purification.

25. A non-specifically amplified natural short-fragment nucleic acid obtained by the method according to method 1.

26. (canceled)

27. A composition comprising the non-specifically amplified natural short-fragment nucleic acid of claim 25.
